(19) United States
(12) Patent Application Publication
(10) Pub. No.: US 2018/0116854 A1
(43) Pub. Date: May 3, 2018

(54) DYNAMIC OVEREXERTION ALERT AND INJURY PROTECTION SYSTEM

(71) Applicant: Chadley Guerrier, Hollywood, FL (US)

(72) Inventor: Chadley Guerrier, Hollywood, FL (US)

(21) Appl. No.: 15/723,761

(22) Filed: Oct. 3, 2017

Related U.S. Application Data
(63) Continuation-in-part of application No. PCT/US16/25939, filed on Apr. 4, 2016.
(60) Provisional application No. 62/142,982, filed on Apr. 3, 2015.

(51) Int. Cl. A61F 5/01; A61B 5/00

(52) U.S. Cl. CPC A61F 5/0123; A61B 5/0492; A61B 5/4836

(57) ABSTRACT

A dynamic overexertion alert and injury protection system and method for monitoring tissue activity surrounding a joint and, some embodiments, automatically supporting the movement of the joint to prevent injuries to and around the joint in response to the monitored activity is implemented through a protective sleeve assembly having a sleeve base, sensor system, control system and locking mechanism. When in place on a target joint, the sensor system operates to assess the activity of tissue surrounding the target knee and generate electrical signals corresponding to the tissue activity while the control system selectively generates alerts and/or causes the locking system the sleeve base to restrict movement of the target joint based on a comparison of assess activity and threshold baselines. Accordingly, the protective sleeve assembly may operate to automatically initiate a protective movement restriction on a target joint wherever a dangerous level of stress is sensed.

### CROSS REFERENCE TO RELATED APPLICATIONS

This application claims the benefit of and incorporates by reference co-pending Patent Cooperation Treaty patent application number PCT/US2016/25939 filed on Apr. 4, 2016, which itself claimed the benefit of U.S. provisional patent application Ser. No. 62/142,982 filed Apr. 3, 2015.

### BACKGROUND OF THE INVENTION

### Field of the Invention

This invention relates generally to an injury alerting and protection system and, more particularly, to a system for alerting of overexertion of ligaments through real time electronic monitoring and protecting ligaments from injury through the selective application of a stress limiting locking mechanism or an assistive force support mechanism.

### Description of the Prior Art

The human condition is frequently centered around our mortality. In a less grave sense, humans are all faced with the inability to disregard the fact that, over time, their bodies degrade from the internal and external stresses applied to our bodies, including through the types of food eaten and sleeping patterns engaged in. Moreover, to compound rejuvenation and slow the degradation, it is common for individuals to stress their body in a different manner; in a manner not unlike the tempering of steel in which one seeks to make their body stronger through exercise. Nonetheless, exercise, as a caveat to its innumerable revitalizing qualities, introduces a harsh reminder of our degrading bodies through increasing the risk of athletic injury.

With respect to exercise, it is well established that the rewards tremendously outweigh the risks of exercise. Yet those risks must be acknowledged; and as individuals make the conscious choice to walk, run, jump, swim, or bike, they must put in jeopardy the ligaments and tendons that act as the gatekeepers of motion that facilitates that invigorating activity known as exercise. To pacify the angst of this decision, external support structures such as braces, sleeves, wraps, and straps may be provided to support the ligaments and tendons. In typical circumstances, however, such external support structures are often provided after the degradation from injury has already ran its course in the form of sprains, strains or even tears.

Attempts have been made to introduce preventative measures for lowering the incidence of injuries to subjects during exercise or physical exertion. Traditionally, neuromuscular training has been employed as a preventative measure for athletes. A problem which still exists, however, is that this method has been measured to only provide a 1.7% decrease in injury susceptibility. Thus, there remains a need for a system and method that can warn of overexertion and may be used prospectively to reduce or remove the probability of injury through the overexertion of ligaments and tendons that frequently occurs during exercise.

When considering non-contact athletic injuries, injuries to the ligaments of the knee occur the most frequently and can be the most debilitating. Similarly, the anterior cruciate ligament (“ACL”), posterior cruciate ligament (“PCL”), lateral collateral ligament (“LCL”), and medial collateral ligament (“MCL”), are all damaged (or completely torn) through hyperextension of the ligament. In the case of each, such hyperextension is generally a result of an excessive load generated from an athletic maneuver (i.e., “overexertion”). Indeed, each ligament has a measurable failure load that, when exceeded, typically will lead to damage: the ACL (front of the knee) is reported to have a failure load of about 2200 Newton, the PCL (back of the knee) has one of about 4000 N, the LCL (outside of the knee), has one of about 750 N, and finally the MCL (inside of the knee) has one of about 650 N. Understanding that the consequences of exceeding the respective failure load for any of these ligaments are dire, there remains a need for a system and method that can generate an alert in response to an application of stress that exceeds the failure load on these respective ligaments, or oven mechanically attempt to counteract such an application of stress, while still generally enabling comfortable and non-obstructive range of motion.

Notably, looking specifically at the ACL, it has been demonstrated that conventional knee braces provide a negligible benefit. Braces may slightly influence knee proprioception, but they do not alter electromyographic firing patterns when compared with the unbraced knee. Furthermore, it has not been demonstrated a decreased incidence of ACL injury in braced versus unbraced athletes. As such, there remains a need for a system and method that provides real-time (dynamic) monitoring of the electromyograph (“EMG”) signal produced by the muscles that introduce tensile stress to the ligaments of interest so as to generate an alert when harmful stress is sensed. It would be helpful for such a dynamic overexertion alert and injury protection system and method to include a locking or support mechanism which is be activated to provide added support when the ligaments of the knee experience forces induced from normal gait, well beneath ultimate strength of the ligaments despite detrimental influence from internal factors that predispose people to injury. It would be additionally desirable for such a dynamic overexertion alert and injury protection system and method to be operable to consider specifics about a user, such as age, gender, height, and weight, when monitoring, alerting, locking or supporting a ligament.

The Applicant's invention described herein provides for a computer system and wearable sleeve that are operative to monitor the electrical activity of muscles surrounding a joint and selectively add support to the joint based on muscle activity. When in operation, the dynamic overexertion alert and injury protection system and method prevents the application of stress above a desired range on connective tissue integral or adjacent to a joint. As a result, many of the limitations imposed by prior art systems and methods are removed.

### SUMMARY OF THE INVENTION

A dynamic overexertion alert and injury protection system and method for monitoring tissue activity surrounding a joint and automatically supporting the movement of the joint to prevent injuries to and around the joint in response to the monitored activity. The dynamic overexertion alert and injury protection system and method comprises in one embodiment a protective sleeve assembly defining a sleeve base, a sensor system, a control system, and a locking mechanism. In other embodiments, the dynamic overexertion alert and injury protection system and method comprises a protective sleeve assembly defining a sleeve base, a sensor system, and a control system. The sleeve base provides a wearable component through which the sensor system, control system and locking mechanism can be held in place on a user and thus may include a plurality of securing straps to facilitate securing it over a target joint, such as a knee. The sensor system defines a plurality of electromyographic pads which monitor and report electrical activity produced by targeted human tissue. The control system communicates with the sensor system and uses inputs from the sensors to selectively operate the locking mechanism and/or generate alerts. The locking mechanism operates to introduce added support to the target joint to discourage movements that may overstress the tendons, ligaments, and other tissue appurtenant the joint.

In operation, when in place on a target knee, the sensor system operate to assess the activity of tissue surrounding the target knee and generate electrical signals corresponding to the tissue activity, while the control system selectively operates the locking mechanism to restrict movement of the target knee and/or generates alerts based on a comparison of assessed activity and threshold baselines. Accordingly, the protective sleeve assembly can automatically initiate a protective movement restriction on a target knee wherever a dangerous level of stress is sensed.

It is an object of this invention to provide a system and method that provides real-time monitoring of the EMG signal produced by the muscles that introduce tensile stress to the ligaments of interest so as to generate an alert when harmful stress is sensed.

It is another object of this invention to provide a system and method which includes a locking and/or support mechanism which is be activated to provide added support when the ligaments of the knee experience forces induced from normal gait added support when the ligaments of a joint experience forces induced from normal gait, well beneath ultimate strength of the ligaments despite detrimental influence from internal factors that predispose people to injury.

It is yet another object of this invention to provide a system and method configurable to consider specifics about a user, such as age, gender, height, and weight, when monitoring, alerting, locking or supporting a ligament.

These and other objects will be apparent to one of skill in the art.

### BRIEF DESCRIPTION OF THE DRAWINGS

FIG. 1 is a top plan view of a protective sleeve assembly of a dynamic overexertion alert and injury protection system and method built in accordance with a first knee embodiment of the present invention in an open configuration.

FIG. 2 is a top plan view of a component liner of a protective sleeve assembly of a dynamic overexertion alert and injury protection system and method built in accordance with a first knee embodiment of the present invention in an open configuration.

FIG. 3 is a top plan view of the slide path of a sliding locking mechanism of an embodiment of a protective sleeve assembly of a dynamic overexertion alert and injury protection system and method built in accordance with a first knee embodiment of the present invention.

FIG. 4 is a top plan view of the slide path of a sliding locking mechanism of an embodiment of a protective sleeve assembly of a dynamic overexertion alert and injury protection system and method built in accordance with a first knee embodiment of the present invention.

FIG. 5 shows an electrical diagram showing the electrical components of a dynamic overexertion alert and injury protection system and method built in accordance with an embodiment of the present invention.

FIG. 6 shows a schematic of simplified circuitry of a dynamic overexertion alert and injury protection system and method built in accordance with an embodiment of the present invention.

FIG. 7 is an exploded back perspective view of a protective sleeve assembly of a dynamic overexertion alert and injury protection system and method built in accordance with a second knee embodiment of the present invention.

FIG. 8 shows exemplary screen shots of a monitoring system application of a protective sleeve assembly of a dynamic overexertion alert and injury protection system and method built in accordance with an embodiment of the present invention.

FIG. 9 is a side elevational view of a biased locking mechanism of a protective sleeve assembly of a dynamic overexertion alert and injury protection system and method built in accordance with an embodiment of the present invention.

FIG. 10 is top plan view of a protective sleeve assembly of a dynamic overexertion alert and injury protection system and method built in accordance with a third knee embodiment of the present invention.

FIG. 11 is top plan view of a protective sleeve assembly of a dynamic overexertion alert and injury protection system and method built in accordance with a third knee embodiment of the present invention shown with the cover of the component housing removed.

FIG. 12 is a bottom plan view of a protective sleeve assembly of a dynamic overexertion alert and injury protection system and method built in accordance with a third knee embodiment of the present invention.

FIG. 13 shows a schematic of simplified circuitry of a dynamic overexertion alert and injury protection system and method built in accordance with a third embodiment of the present invention.

### DETAILED DESCRIPTION OF THE INVENTION

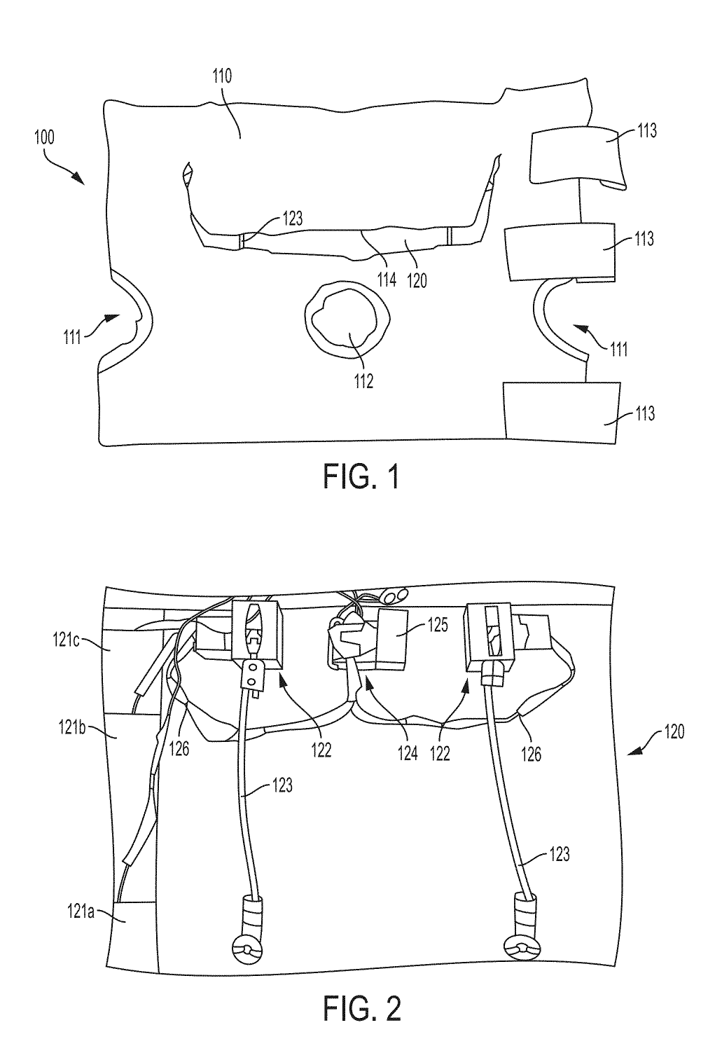

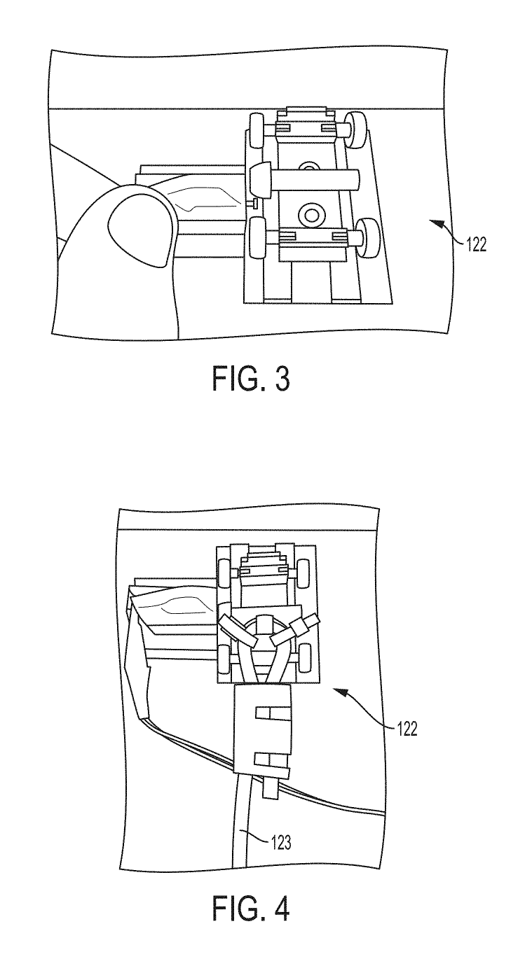

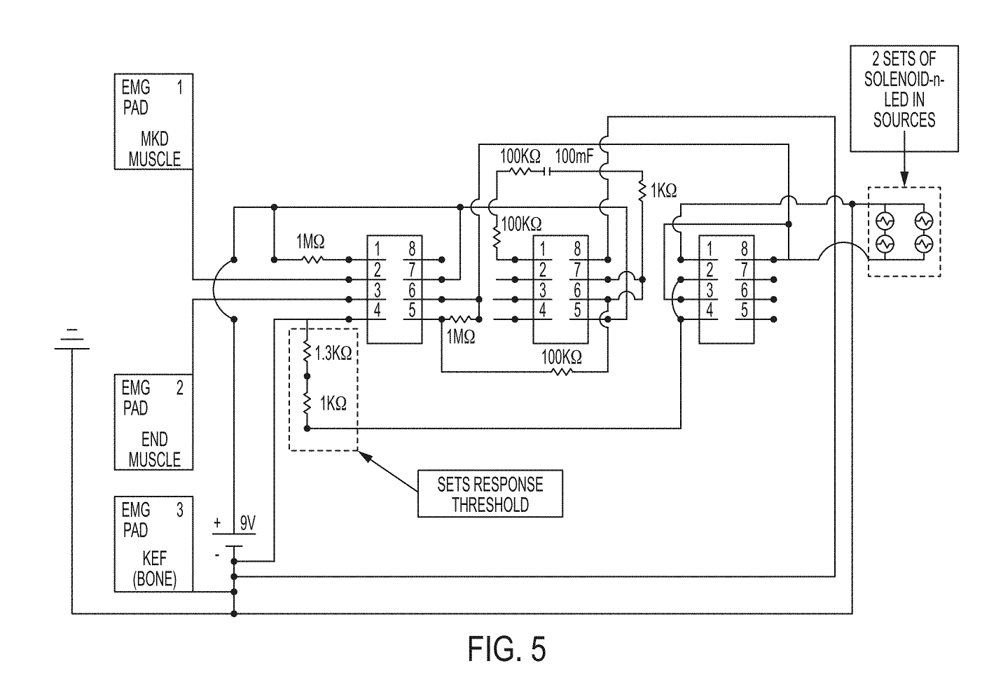

Referring now to the drawings and in particular FIGS. 1, 2, 3, 4, 5 and 6, in one embodiment a dynamic overexertion alert and injury protection system and method includes protective sleeve assembly defined by a modified knee sleeve 100 having a sleeve exterior 110 and a component liner 120. The modified knee sleeve 100 defines a wrap around wearable knee sleeve that includes on its sleeve exterior 110 a front knee section 111 for receiving the front of a wearer's knee, a back knee section 112 for receiving the back of a wearer's knee, a plurality of securing straps 113 for selectively fixing the modified knee sleeve 100 in place on a wearer's knee, and an access flap 114 for selectively exposing the component liner for servicing.

It is contemplated that to supplement the securing straps 113, compression bands (not shown) may be employed for retaining the modified knee sleeve 100 on a wearer. The amount of compression to be used will determined by the radius of the wearer's upper thigh region. As it is appreciated that the capillary closing pressure is 32 mmHg (0.618 psi), that will be used as the upper limit of the applied pressure as exceeding this limit can cause tissue damage. It is additionally appreciated that at 15 mmhg (0.2895 psi), blood flow velocity was increased 5-fold and this added pressure was capable of decreasing muscle recovery time after high intensity exercise. Accordingly, using this pressure (coupled with the empirically determined static coefficient of friction for human skin: 0.11), it can be calculated that the desired pressure applied to the skin can be given by the equation: pressure against skin=2π(radius)(2″ thickness)(0.2895 psi)(0.11) (given the thickness of the compressive region at the top and bottom of the sleeve are 2 inches each).

In one embodiment, the component liner 120 houses a sensor system, control system and locking mechanism of the modified knee sleeve 100, with a restriction implement integrated with the modified knee sleeve 100. In alert only embodiments, the component liner houses only the sensor system and control system of the modified knee sleeve.

In the illustrated embodiment, three electromyographic pads, collectively, 121 (shown inserted in their respective places in the component liner 120) define the sensor system, two sliding locking mechanisms 122 define the locking mechanism, support structures 123 which each correspond to one of the sliding mechanisms define the restriction implement, and an electronic components control member 124 and a power source 125 define the control system. In one embodiment, the components control member 124 includes an integrated precision gain differential amplifier, an operational amplifier, and a dual comparator (or “differential” comparator).

In operation, the modified knee sleeve 100 is worn on the knee of a user in a manner similar to a conventional knee sleeve. When in place on the user's knee, the precision gain differential amplifier receives signals from the electromyographic pads 121, which by virtue of their location in the component liner 120 are be placed in desired locations against the skin of the wearer. In this regard, the electromyographic pads 121 operate as surface EMG recording electrodes. In one implementation, one electromyographic pad 121a is placed for reference against the bone near the ligaments and/or tendons that are to be protected, one electromyographic pad 121b is placed near the end of the muscle that applies tension to the ligament of interest, and one electromyographic pad 121c is placed against the middle of that same muscle and/or muscle group.

The signals from the electromyographic pads 121 are amplified by the use of the operational amplifier and compared against a predetermined threshold using a ratio of resistors directed through the differential comparator. When the differential comparator reads a signal (that increases resistance) greater than that set by the ratio of resistors, the power from the power source 125 is supplied to the sliding locking mechanism 122.

Due to the use of the three electromyographic pads 121 in the specified areas, the predetermined threshold value that is used to determine whether a dangerous level of stress is present on the ligaments of the targeted joint can be a single value assigned to each electromyographic pads 121, or a composite threshold value, which is defined by a threshold for at least two of the electromyographic pads 121. By operation of the composite threshold value, even if none of the values of the electromyographic pads 121 meet or exceed their individual threshold value, if the value of one of the electromyographic pads 121 meets a defined value (again, that is lower than the individual threshold) at the same time that the value of another of the electromyographic pads 121 meet a defined value, the composite threshold value may be met or exceeded.

The sliding locking mechanisms 122 each define miniature solenoids operative to restrict the movement of its corresponding support structure 123 by interrupting an integral slide path linked to the support structure 123. This interruption of the normally free sliding path occurs when electrical power is supplied to the locking mechanism. In the illustrated embodiment, the support structure 123 defines a rigid steel cable anchored to a distal location on the component liner 120. It is contemplated the support structures 123 are anchored to a location that will inhibit further elevation of muscle activity against a target ligament (targeted for protection) when the sliding path is interrupted. As such, the sliding locking mechanisms 122 cause the support structures 123 to provide added support through the interruption of the sliding path, which occurs when elevated muscle activity is experienced.

In one embodiment, an LED 126 is integrated with each locking mechanism 122 so as to visually depict its activation (interruption of the sliding path).

In one embodiment, an Texas Instruments™ INA106 is used as the precision gain differential amplifier, a Texas Instruments™ TL072 Low-Noise JFET-Input Operational Amplifier is used as the operational amplifier, a Fairchild semiconductor KA393 is used the differential comparator, and 9 V battery is used as the power source 125.

It is contemplated that in one embodiment, a wireless transceiver is integrated with the control system of the modified knee sleeve 100, thereby enabling the activity recorded by the electromyographic pads 121 to be transmitted to other computer devices.

It is contemplated that the alert only embodiments condition and compares the signals from the electromyographic pads in the same manner as described above, but to generate a visual and/or audible alert as opposed to energizing a locking mechanism. It is appreciated that the signal values and the alerts (collectively, results) may be tracked by to control system of the modified knee sleeve and/or transmitted to a remote device for tracking or alert generation. Such results may be associated with specifics about a wearer, such as age, gender, height, and weight, and then used to develop predictive models.

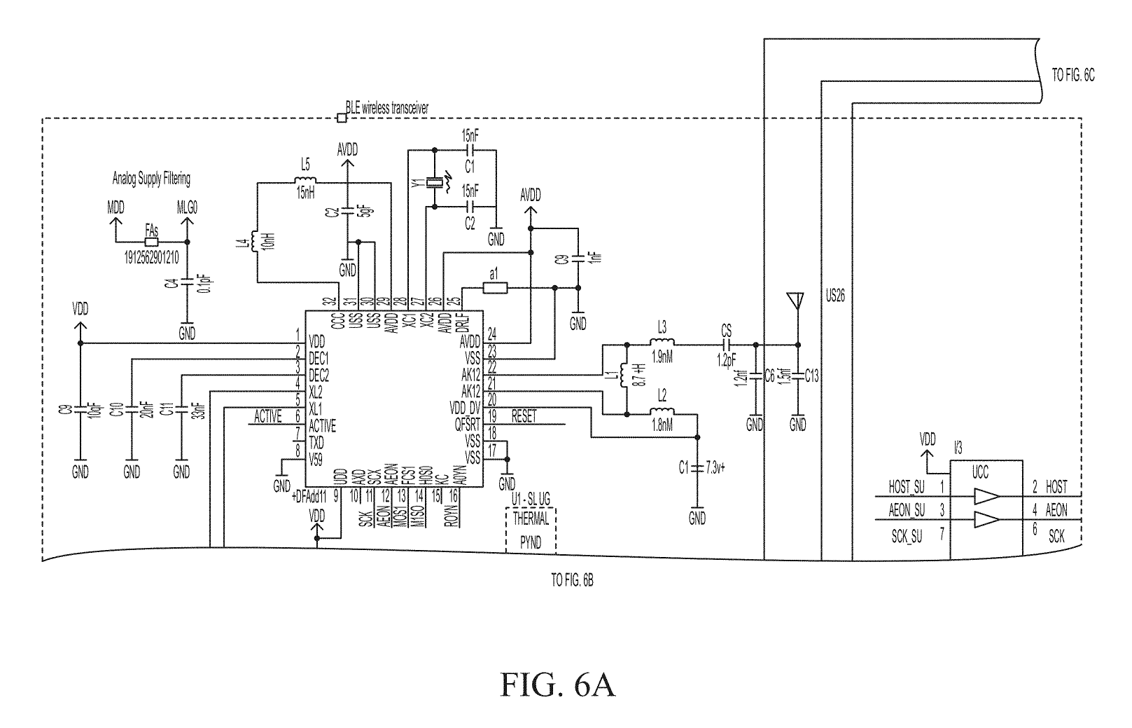

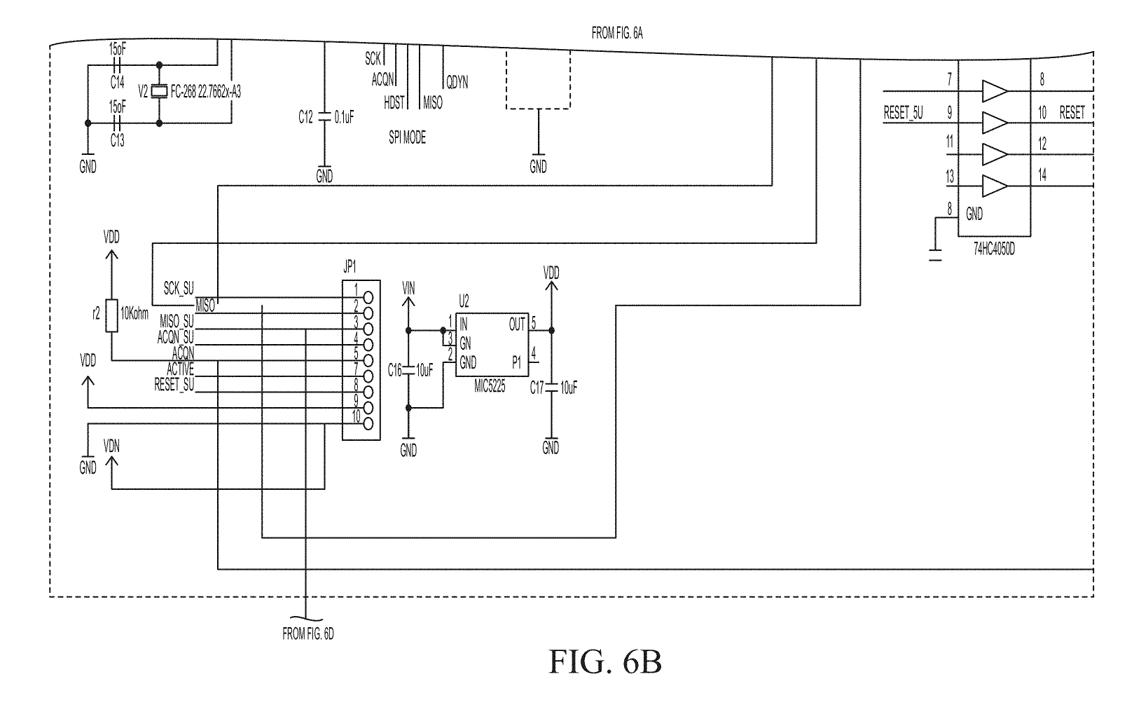

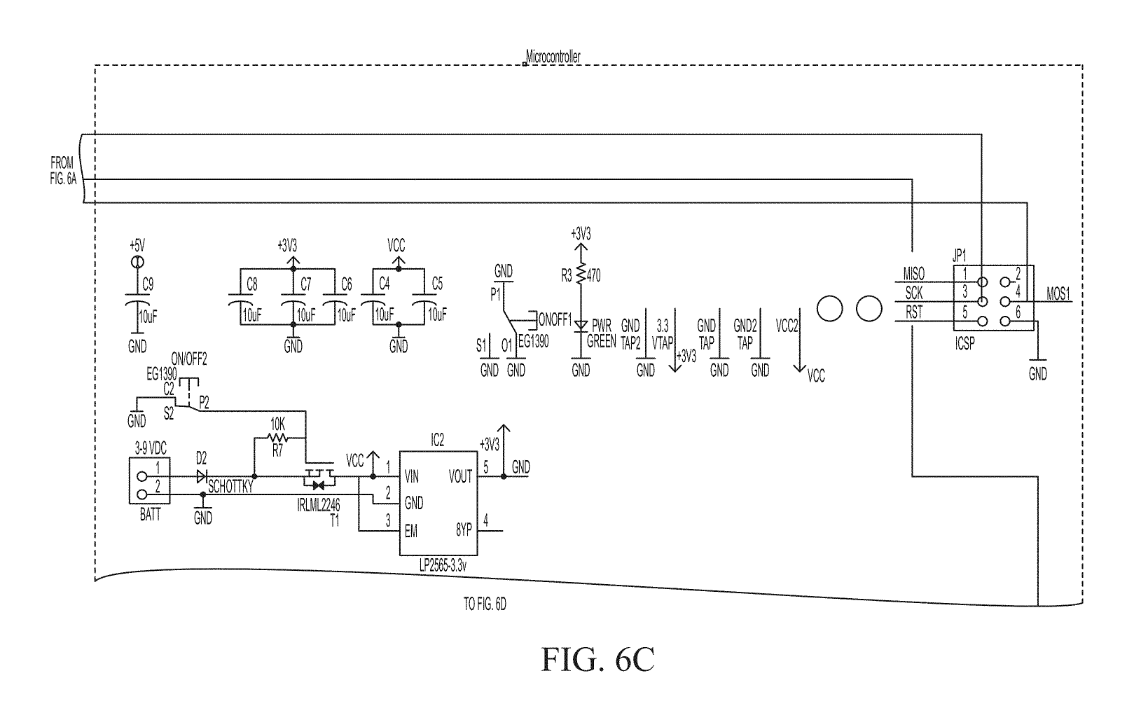

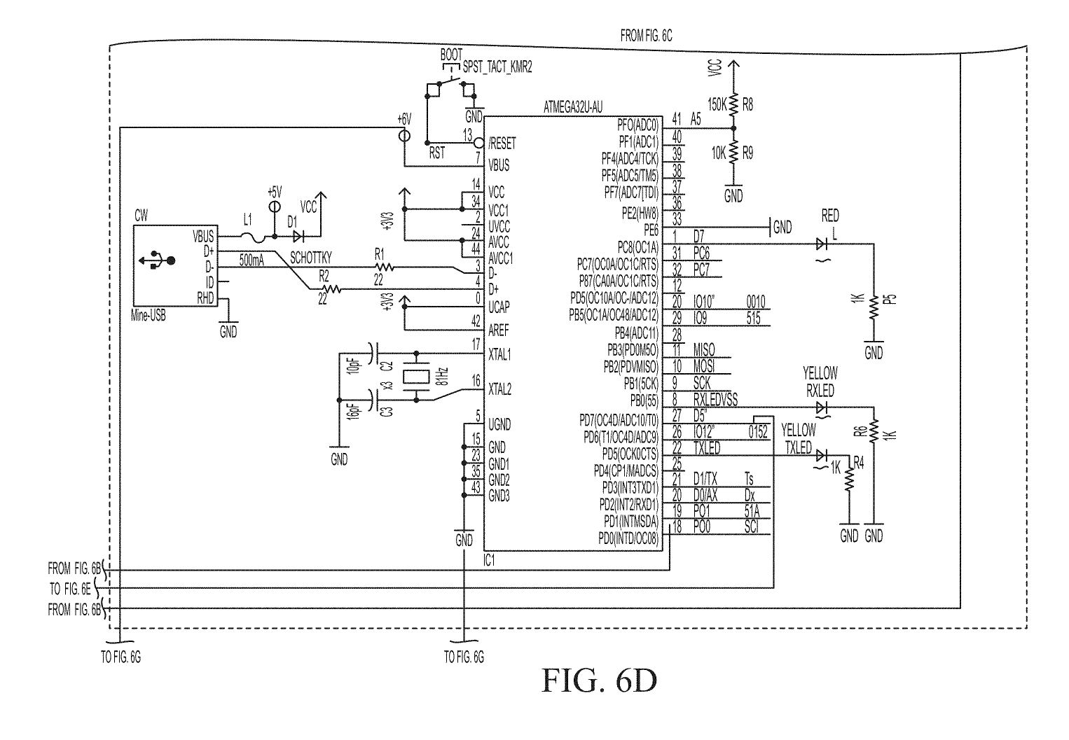

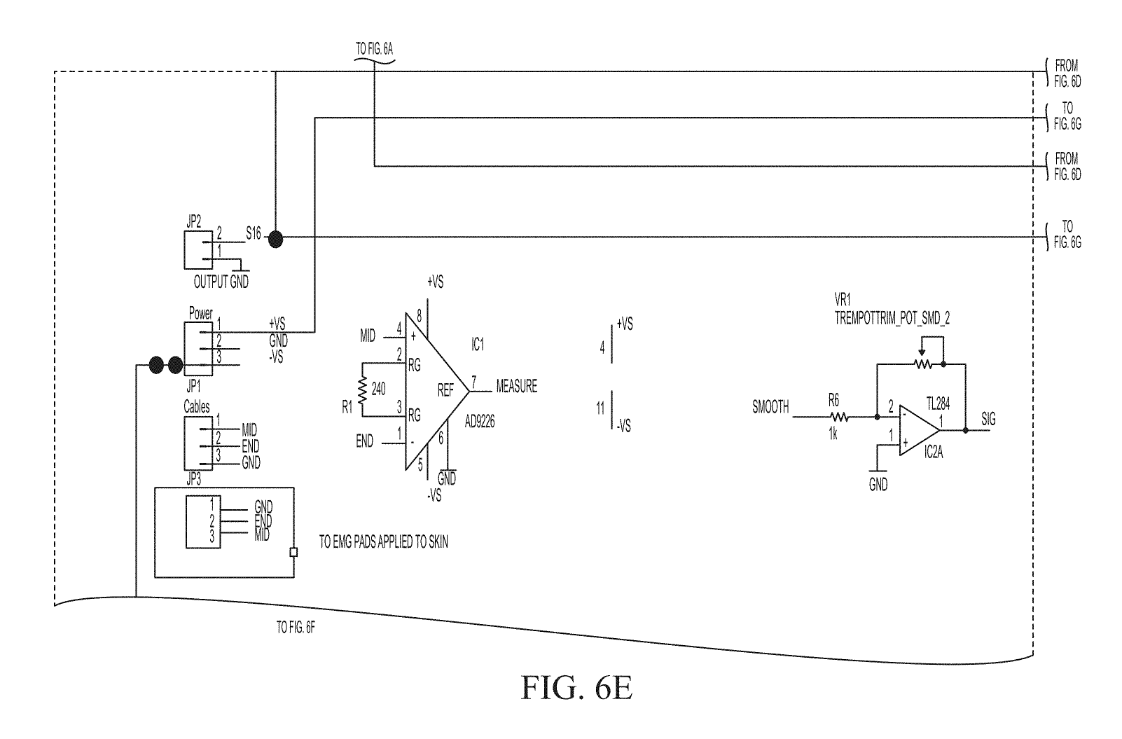

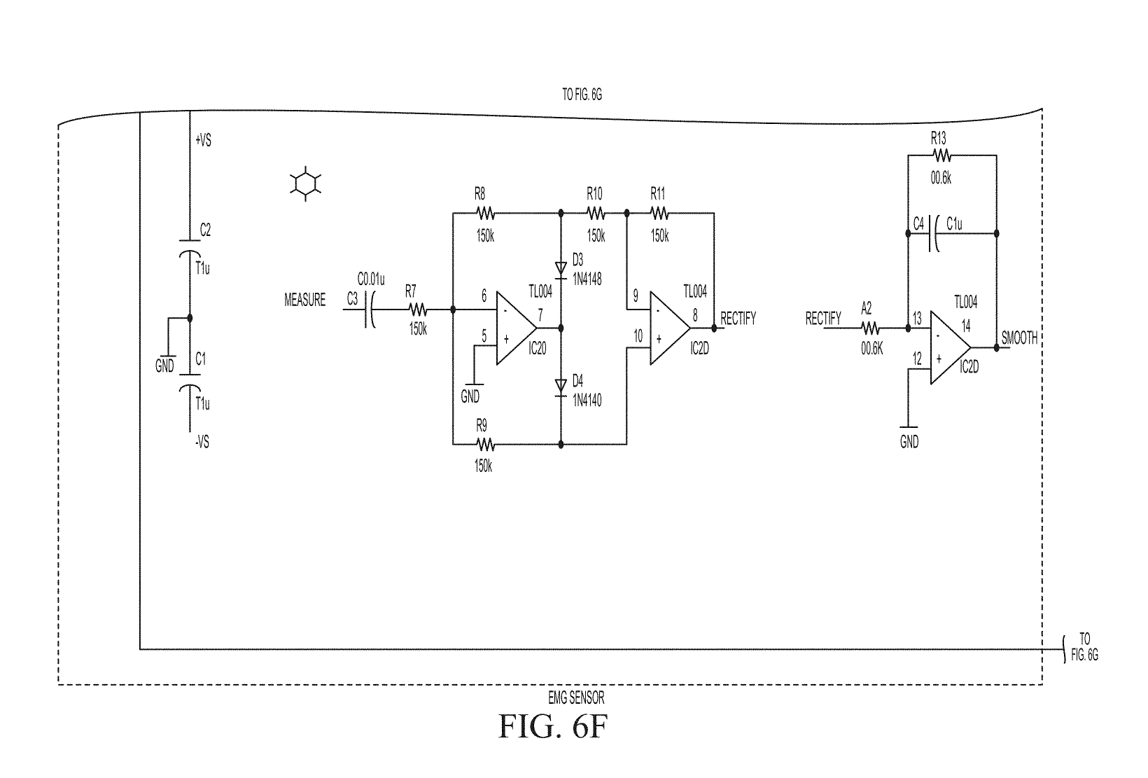

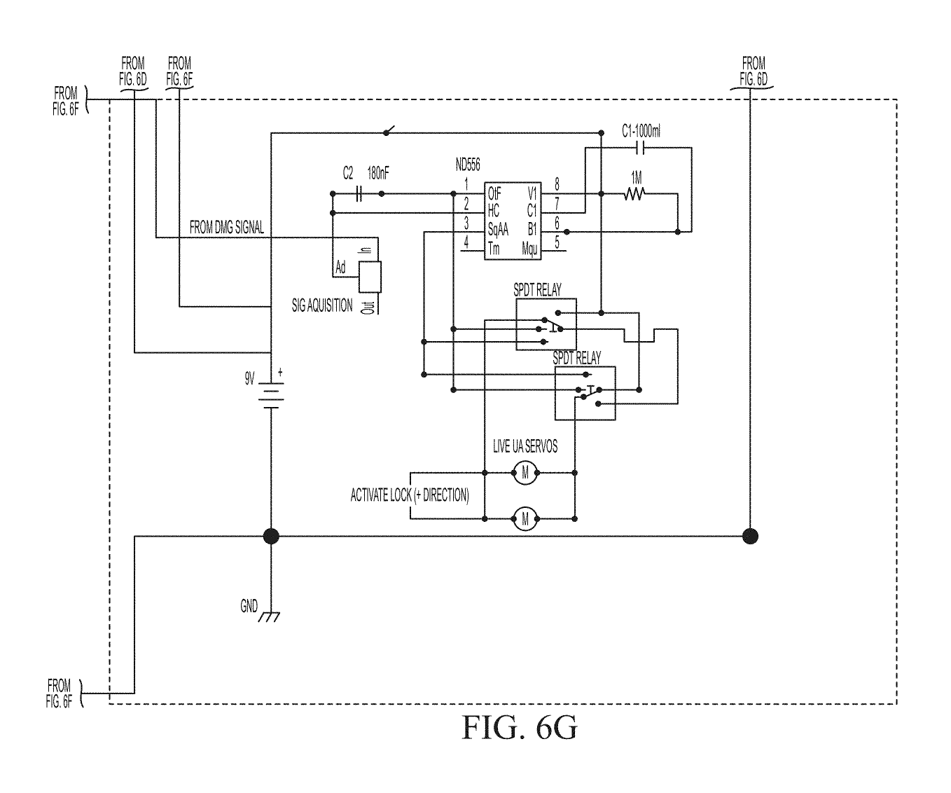

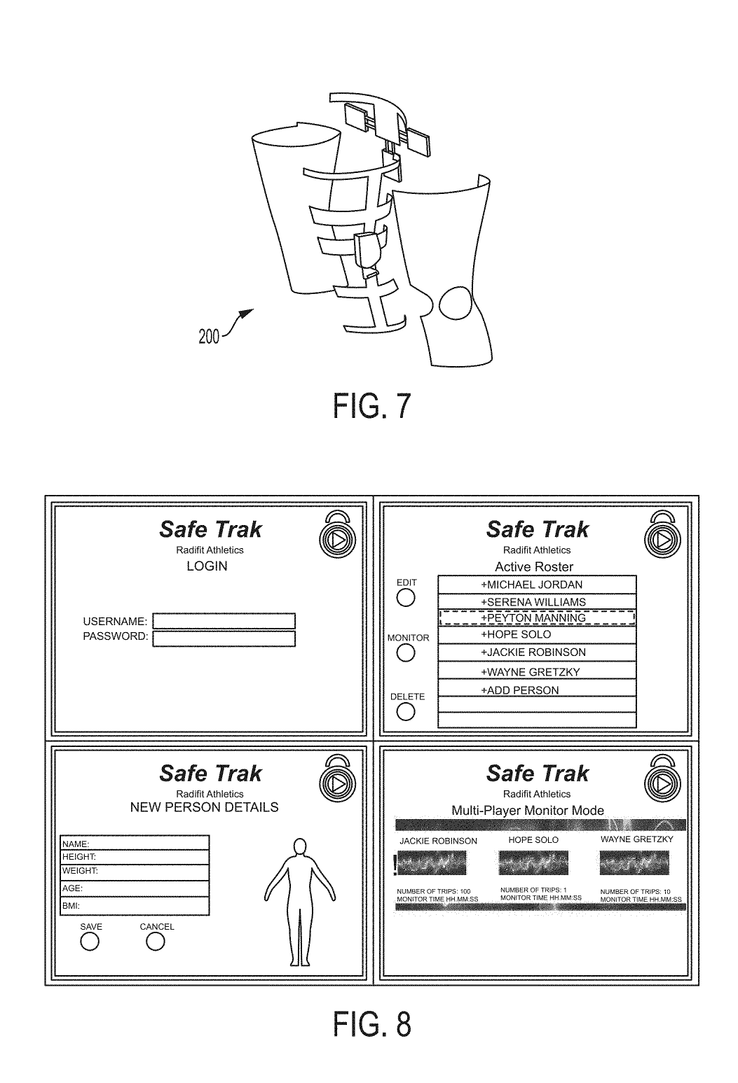

Referring now to FIGS. 6 and 7, a simplified version of a modified sleeve 200 is shown as a flexible PCB sandwiched between two breathable fabric layers and non-slip material to provide compression, and strong adhesion to the skin during use. The schematic in FIG. 6 illustrates the simplified circuitry to be printed on such an embedded flexible PCB. This PCB will use 2 Single Pole Double Throw relays (or alternatively a single Double Pole Double Throw relay) to activate/deactivate the locking mechanism (by reversing the direction of the linear servos).

Referring now to FIG. 8, when wireless transmission of electromyographic pads 121 is enabled, a monitoring system embodied in instructions contained in computer software may be additionally employed to facilitate real time review (and review at a subsequent time) of the stresses a wearer's ligaments is being (or has been) subjected to. A mobile software may thus be deployed to prospectively assess threats which may be present for a wearer of the modified knee brace. Exemplified herein are screenshots of the showing a login screen, a personal detail screen, a roster screen, and a multi-player monitor function up to 3 people can be monitored at once. In the tracking software, the exclamation point next to “Jackie Robinson's” monitor (as a hypothetical) signifies that this user has passed what the software, based on his details, determined is a safe number of locking mechanism trips (slide path interruptions) over a specific duration of time and should be rested.

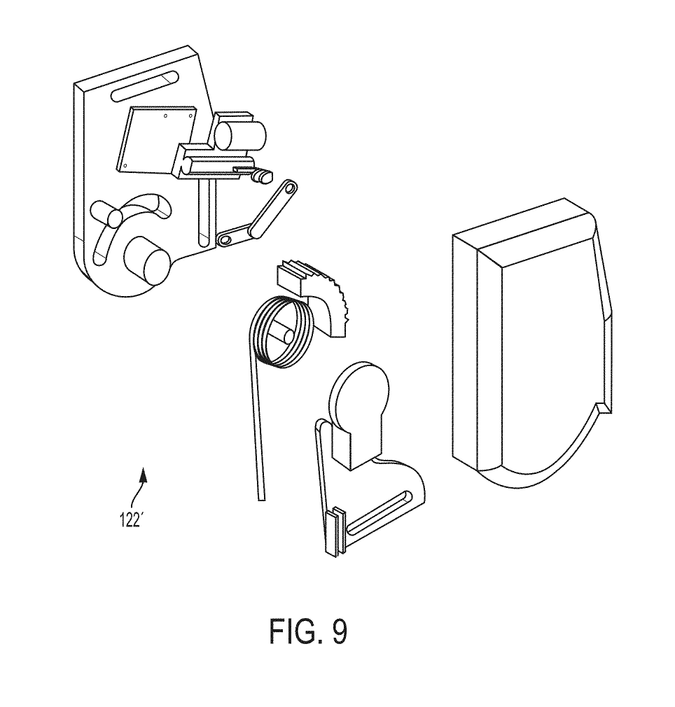

Referring now to FIG. 9, an alternate embodiment of a locking mechanism 122′ is shown having a small nylon linear servo motors and torsion springs to activate/release the locking response as opposed to solenoids.

It is contemplated that using the concepts applied to the ligaments of the knee, the EMG pads, the responsive circuit with the solenoids, the slide path that is to be interrupted, as well as the wearable sleeve with retention features, the instant application can be applied across different ligaments in the body with modified geometry and quantities of the same components used in the knee sleeve prototype as the ligaments of the body are, from athletic maneuvers, subject to injury through hyperextension/overexertion.

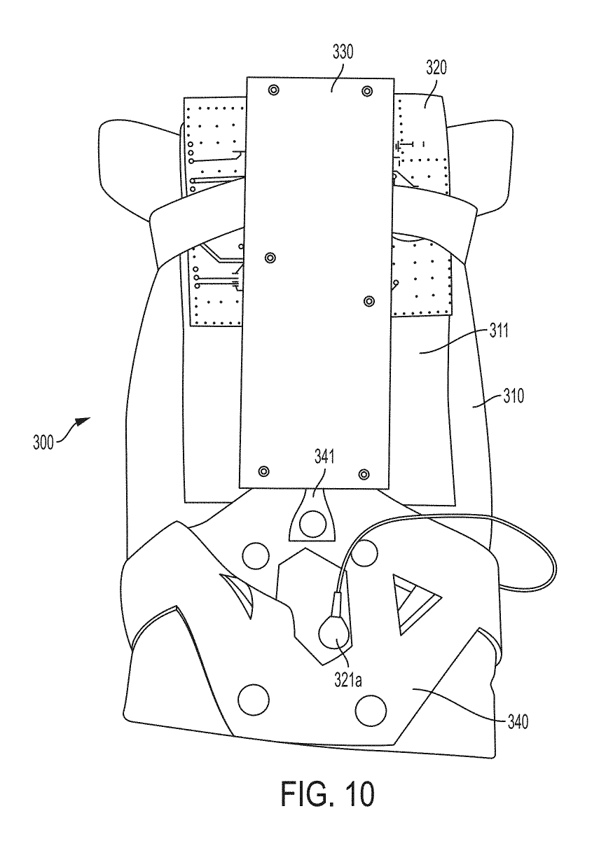

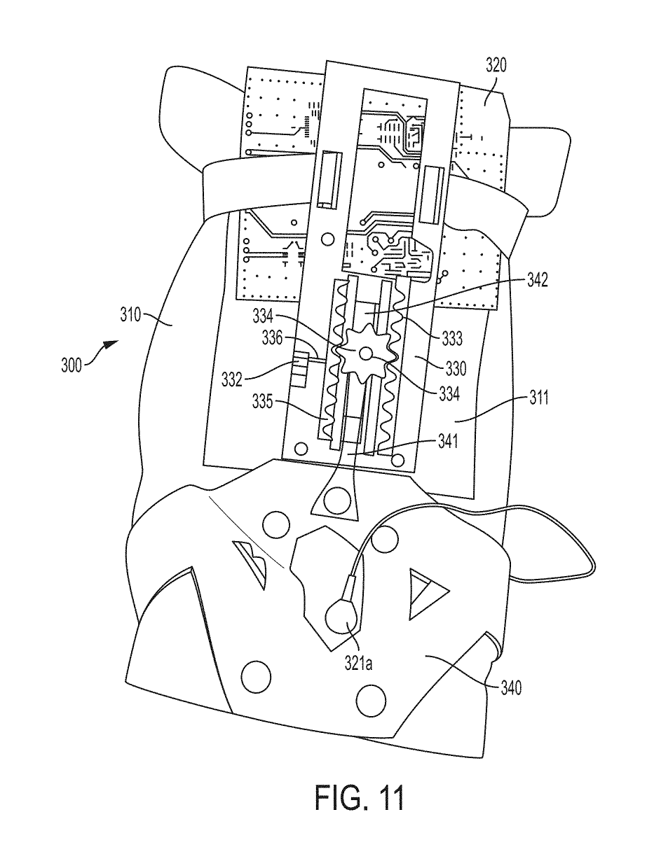

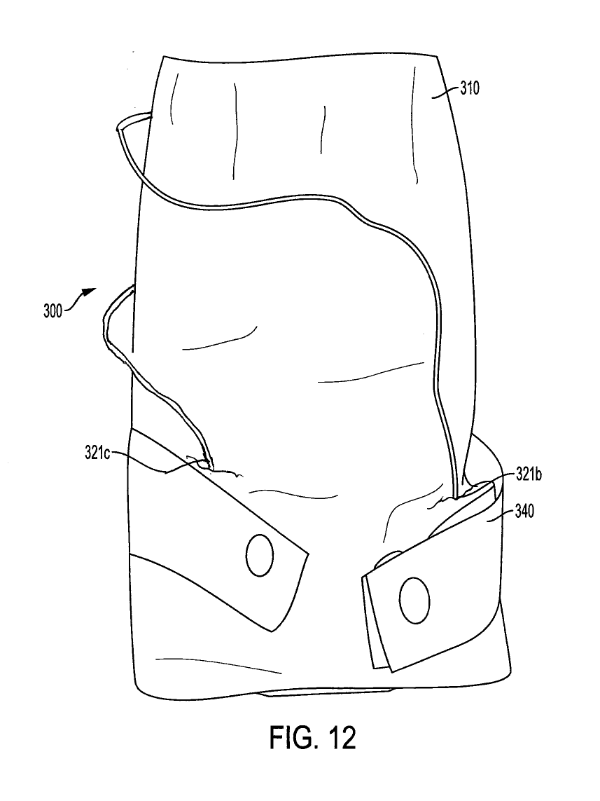

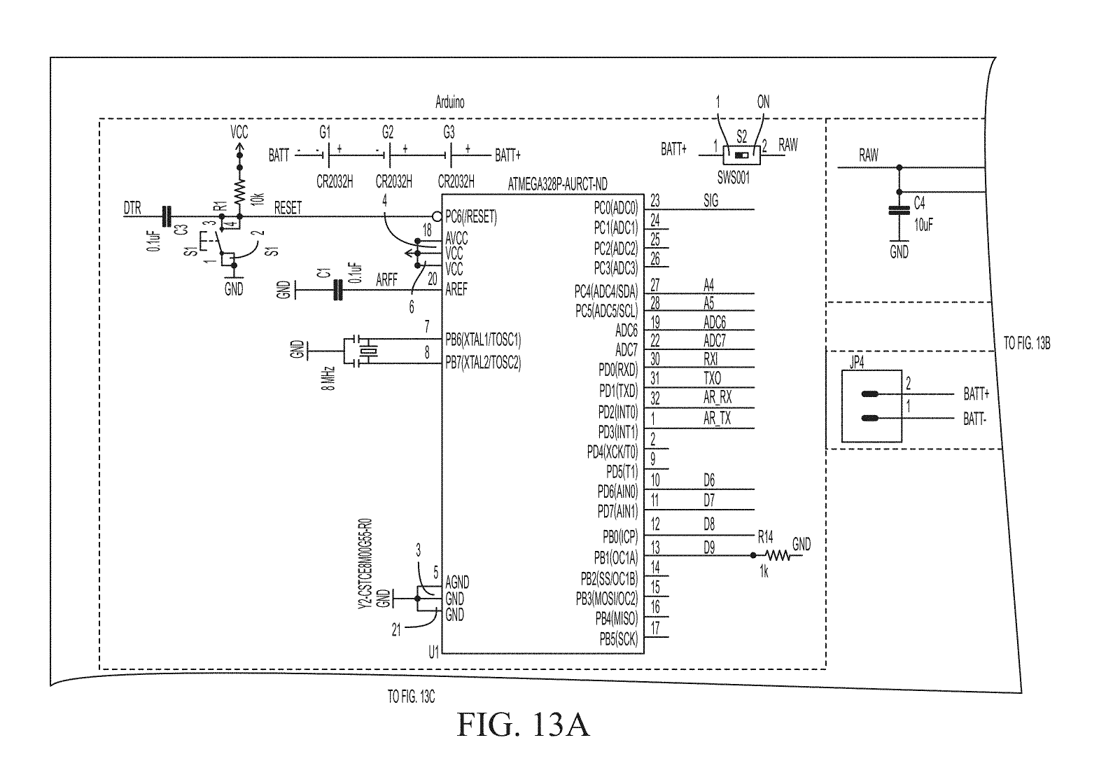

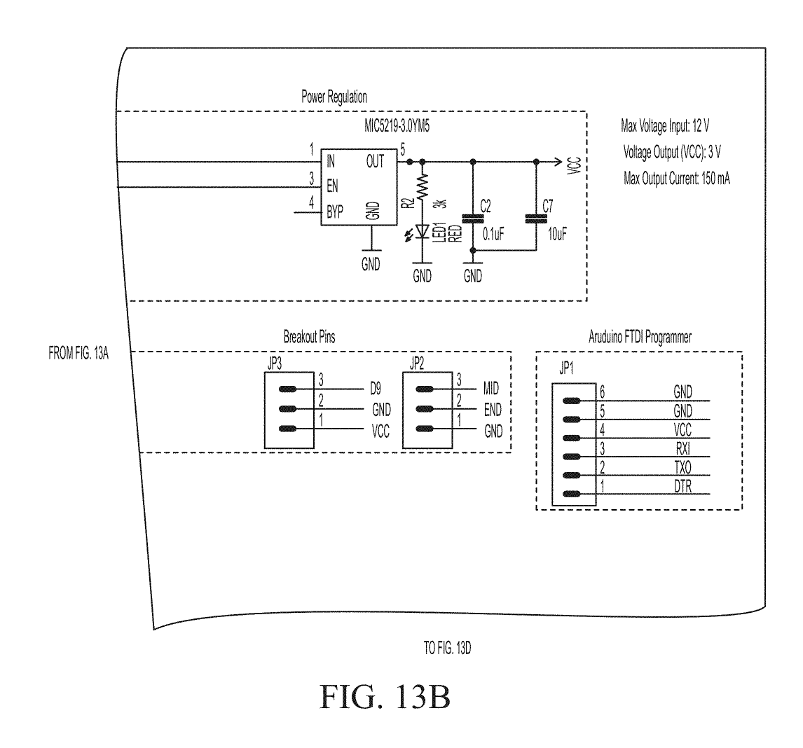

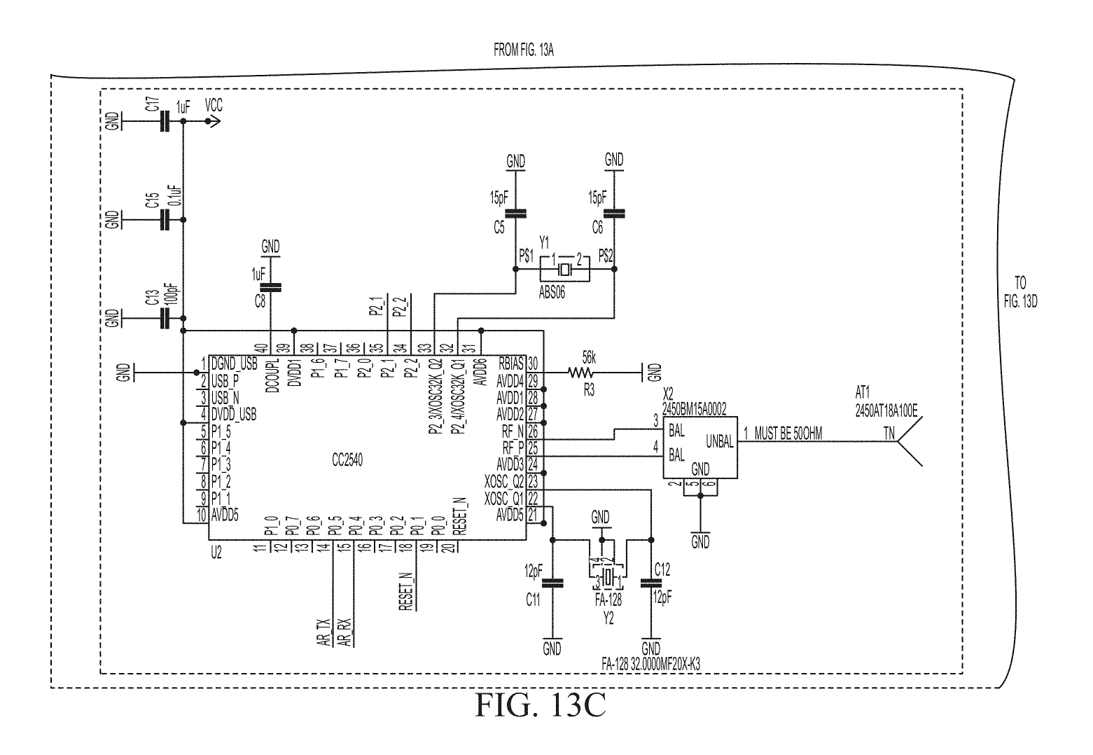

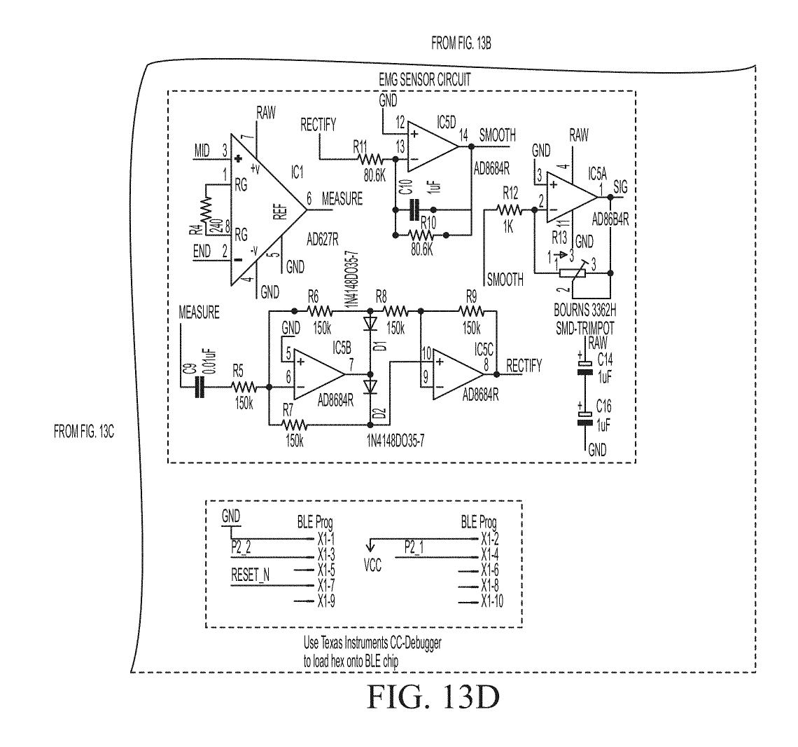

Referring now to FIGS. 10, 11, 12, and 13, in another embodiment, the dynamic overexertion alert and injury protection system and method includes protective sleeve assembly defined by a modified knee sleeve 300 having a wearable sleeve base 310, a components control member defining a flexible printed circuit board 320, a component housing 330, and a support structure 340. The component housing 330 is coupled with the sleeve base 310 through a securing loop 331 such that the component housing 330 may be held in place above a wearer's knee, adjacent to the thigh. The support structure 340 is attached to the sleeve base 310 such that the support structure 340 may be held in place over a wearer's knee. In this regard, the hinge action created by a wearer bending their knee causes the support structure 340 and component housing 330 to progressively move apart as the knee bends.

The circuit board 320 and a foam layer 311 are wedged between the component housing 330 and sleeve base 310, with the circuit board 320 electrically connected to three electromyographic pads, collectively, 321 positioned in the sleeve base 310, a motor 332 housed in the component housing 330, and an onboard power source (not shown), housed in the component housing 330.

Disposed inside the component housing 330 is the motor 332, defined in one embodiment as a servo motor, and a sprocket and track assembly. The sprocket and track assembly includes a fixed track 333, a sprocket 334 and a moving track 335 and operates with the such that the sprocket 334 is generally able to move up and down the fixed track 333 with the moving track 335 moving along with the sprocket 334. The motor 332 operates to selectively engage a locking pin 336 with the moving track 335 so as to lock the moving track 335 and sprocket 334 in their instant location.

Connected to the sprocket 333 is a first connector 341 defining in one embodiment a substantially rigid, vinyl strap and a second connector 342 defining in one embodiment an elastic, rubber strap. The first connector 341 attaches to the sprocket 333 at one end, extends directly to the support structure 340, and attaches to the proximal (relative to the component housing 330) portion support structure 340 at its opposite end. The second connector 342 attaches to the sprocket 333 at one end, passes underneath the component housing 330 and support structure 340, and connects to the proximal (relative to the component housing 330) portion support structure 340 at its opposite end. In this regard, as long as the sprocket 333 is not locked by action of the motor 332, the sprocket 333 and first connector 341 may move up and down freely as a wearer's knee bends. On the other hand, when the sprocket 333 is locked in place, the first connector 341 tensions the support structure 340, thereby creating resistance which opposes the bending of the wearer's knee and adds support to the wearer's knee. The second connector 342 operates to keep the sprocket 333 at the most distal point relative to the support structure 340 at all times, so that whenever the sprocket is locked, it is positioned to provide the maximum amount of resistance for the present knee position.

In this embodiment the electromyographic pads 321 (shown inserted in their respective places in the sleeve base 310) define the sensor system, the support structure 340 defines the restriction implement, the sprocket and track assembly, motor, first connector and second connector define the locking mechanism, and the circuit board 320 and power source define the control system. It is contemplated that the circuit board 320, as the components control member may include components for amplifying signals from the sensor system, conditioning them for comparison, and comparing them against a predetermined threshold.

In operation, the modified knee sleeve 300 is worn on the knee of a user in a manner similar to a conventional knee sleeve with one electromyographic pad 321a placed for reference against the femur bone near the ligaments and/or tendons that are to be protected, one electromyographic pad 321b is placed near the lateral head of the gastrocnemius muscle, and one electromyographic pad 321c is placed against the medial head of the gastrocnemius muscle.

The signals from the electromyographic pads 121 are amplified by the use of the operational amplifier and compared against a predetermined threshold using a ratio of resistors directed through the differential comparator. When the differential comparator reads a signal (that increases resistance) greater than that set by the ratio of resistors, the power from the power source 125 is supplied to the sliding locking mechanism 122.

Thus, in one embodiment, entire dynamic overexertion alert and injury protection system employs:

(A) A knee sleeve that uses a series of straps and nylon bands specifically oriented to provide a counter force against 3 main causes of ACL injuries: (1) Adductor torsion about the tibiofemoral joint (joint between the upper and lower leg); (2) Anterior tibial translation (when lower leg bone moves slightly forward); and (3) Excessive tensile force applied to the patellar tendon from the quadriceps. It is contemplated that this counterforce will reduce the stress applied to the ACL, thus preventing the ligament from tearing.

(B) A locking mechanism on the knee sleeve to introduce added support of the oriented straps/band to discourage a force that may lead to an overstressed (injured) ACL. The locking mechanism will be activated when a load corresponding to 45% of the failure load of the ACL (to be conservative) is detected and remain activated for the duration of the maneuver (with a minimum of 100 ms).

(C) A real-time monitoring application that will receive transmitted muscle activity data of the gastrocnemius muscle (calf muscle) and use that data to recommend rest time of the hamstring muscle specifically.

In such an embodiment, treatment may include a focus on recommending rest of the hamstring muscle because the hamstring protects the ACL ligament by discouraging anterior tibial translation (ATT) during intense maneuvers. By following the recommended rest alerts for the hamstring, the user decreases the likelihood of the hamstring insufficiently protecting against ATT.

Moreover, in such an embodiment it contemplated that it may be beneficial to relate calf muscle activity to hamstring activity because during normal gait, isotonic cycling and landing (from a high jump), they follow similar excitation patterns, a diagram of the drop landing pattern is shown below.

It is appreciated that each embodiment of the modified knee sleeve discussed above may be employed as an alert only embodiment that includes sensing and control components, a locking embodiment that includes sensing, locking and control components, or an assistive embodiment that includes sensing, control and support components, with the support components operative to add assistive force to the joint for those suffering from some muscle atrophy or the like.

The instant invention has been shown and described herein in what is considered to be the most practical and preferred embodiment. It is recognized, however, that departures may be made therefrom within the scope of the invention and that obvious modifications will occur to a person skilled in the art.

What is claimed is:

1. A wearable dynamic overexertion alert and injury protection, comprising
a knee sleeve adapted to be worn by a user so as to cover a knee joint of the user;
a plurality of electromyographic sensors integral with said knee sleeve, wherein said each of said electromyographic sensor is positioned to assess muscle activity adjacent to the knee joint of the user; and
a control device integral with said knee sleeve and adapted to receive an input signal from each of the plurality of electromyographic sensors, wherein each respective input signal is related to the activity of the muscle being assessed by one of the plurality of electromyographic sensors; and
said control device additionally adapted to process the input signals, and, in the event a value of any of the respective input signals exceeds a predetermined threshold value for the source electromyographic sensor for the respective input signal, selectively generate an alert indicative of a dangerous level of stress.

2. The wearable dynamic overexertion alert and injury protection system of claim 1, additionally comprising:
at least one restriction implement integral with said knee sleeve and configured to selectively restrict the movement of the knee sleeve in a manner which inhibits further elevation of muscle activity assessed by at least one of the plurality of electromyographic sensors; and
a locking mechanism operatively connected to said control device, wherein said locking mechanism is configured to cause the at least one restriction implement to restrict the movement of the knee sleeve when actuated by the control device.

3. The wearable dynamic overexertion alert and injury protection system of claim 2, wherein said control device is configured to selectively actuate the locking mechanism by activating a motor.

4. The wearable dynamic overexertion alert and injury protection system of claim 2, wherein:
said restriction implement defines an elongated member having a first end connected to the locking mechanism and a second end fixed to a distal anchor point on the knee sleeve; and
the actuation of the locking mechanism is defined by the locking of the first end in a fixed position on the locking mechanism.

5. The wearable dynamic overexertion alert and injury protection system of claim 2, wherein the actuation of the locking mechanism is defined by the locking of a rigid connector which connects the locking mechanism and the support structure.

6. The wearable dynamic overexertion alert and injury protection system of claim 1, wherein said control device includes a differential amplifier which configures the control device to receive the input signals from the electromyographic sensors.

7. The wearable dynamic overexertion alert and injury protection system of claim 1, wherein said control device is adapted to compare each input signal against the predetermined threshold value for the source electromyographic sensor for the respective input signal using a differential comparator, thereby processing the at least one input signal.

8. The wearable dynamic overexertion alert and injury protection system of claim 7, wherein said control device additionally includes an operational amplifier to amplify the input signals from the electromyographic sensors, thereby processing the at least one input signal.

9. The wearable dynamic overexertion alert and injury protection system of claim 1, wherein:
said plurality of electromyographic sensors defines at least a first electromyographic sensor positioned in said knee sleeve so as to enable assessment of an end of a muscle that applies tension to a target tissue and a second electromyographic sensor positioned in said knee sleeve so as to enable assessment of an interior portion of a muscle that applies tension to the target tissue; and
said target tissue defines at least one of a target ligament and a target tendon.

10. The wearable dynamic overexertion alert and injury protection system of claim 9, wherein said at least one electromyographic sensor additionally includes a third electromyographic sensor positioned in said knee sleeve so as to enable a reference assessment near at least one bone adjacent to the target tissue.

11. The wearable dynamic overexertion alert and injury protection system of claim 1, wherein, in the event the value of a plurality of the respective input signals together exceed a predetermined composite threshold value for the respective source electromyographic sensors, selectively generate the alert indicative of a dangerous level of stress.

12. A wearable dynamic overexertion alert and injury protection, comprising:
a knee sleeve adapted to be worn by a user so as to cover a knee joint of the user;
a plurality of electromyographic sensors integral with said knee sleeve, wherein said plurality of electromyographic sensors defines at least a first electromyographic sensor positioned in said knee sleeve so as to enable assessment of an end of a muscle that applies tension to a target tissue and a second electromyographic sensor positioned in said knee sleeve so as to enable assessment of an interior portion of a muscle that applies tension to the target tissue;
a control device integral with said knee sleeve and adapted to receive an input signal from each of the plurality of electromyographic sensors, wherein each respective input signal is related to the activity of the muscle being assessed by one of the plurality of electromyographic sensors; and
said control device additionally adapted to process the input signals, and, in the event a value of any of the respective input signals exceeds a predetermined threshold value for the source electromyographic sensor for the respective input signal or the value of a plurality of the respective input signals together exceed a predetermined composite threshold value for the respective source electromyographic sensors, selectively generate an alert indicative of a dangerous level of stress.

13. The wearable dynamic overexertion alert and injury protection system of claim 12, wherein said target tissue defines at least one of a target ligament and a target tendon.

14. The wearable dynamic overexertion alert and injury protection system of claim 12, wherein said at least one electromyographic sensor additionally includes a third electromyographic sensor positioned in said knee sleeve so as to enable a reference assessment near at least one bone adjacent to the target tissue.

15. The wearable dynamic overexertion alert and injury protection system of claim 12, additionally comprising:
at least one restriction implement integral with said knee sleeve and configured to selectively restrict the movement of the knee sleeve in a manner which inhibits further elevation of muscle activity assessed by at least one of the plurality of electromyographic sensors; and
a locking mechanism operatively connected to said control device, wherein said locking mechanism is configured to cause the at least one restriction implement to restrict the movement of the knee sleeve when actuated by the control device and said control device is configured to selectively actuate the locking mechanism by activating a motor.

16. The wearable dynamic overexertion alert and injury protection system of claim 15, wherein:
said restriction implement defines an elongated member having a first end connected to the locking mechanism and a second end fixed to a distal anchor point on the knee sleeve; and
the actuation of the locking mechanism is defined by the locking of the first end in a fixed position on the locking mechanism.

17. The wearable dynamic overexertion alert and injury protection system of claim 15, wherein the actuation of the locking mechanism is defined by the locking of a rigid connector which connects the locking mechanism and the support structure.

18. The wearable dynamic overexertion alert and injury protection system of claim 12, wherein said control device includes a differential amplifier which configures the control device to receive the input signals from the electromyographic sensors.

19. The wearable dynamic overexertion alert and injury protection system of claim 12, wherein said control device is adapted to compare each input signal against the predetermined threshold value for the source electromyographic sensor for the respective input signal using a differential comparator, thereby processing the at least one input signal.

20. The wearable dynamic overexertion alert and injury protection system of claim 19, wherein said control device additionally includes an operational amplifier to amplify the input signals from the electromyographic sensors, thereby processing the at least one input signal.
